(19) United States
(12) Patent Application Publication
(10) Pub. No.: US 2020/0131054 A1
(43) Pub. Date: Apr. 30, 2020

(54) FILTERING DEVICE HAVING UV LIGHT

(71) Applicant: XIAMEN FILTERTECH INDUSTRIAL CORPORATION, XIAMEN (CN)

(72) Inventors: JIA-JIA LIN, Xiamen (CN); WEI-QIANG XU, Xiamen (CN)

(21) Appl. No.: 16/662,030

(22) Filed: Oct. 24, 2019

(30) Foreign Application Priority Data
Oct. 31, 2018 (CN) 201821781383.0

(51) Int. Cl. C02F 1/32; C02F 1/00; A61L 2/10

(52) U.S. Cl. CPC C02F 1/32; C02F 1/003; C02F 2201/002; C02F 2201/32; A61L 2/10

(57) ABSTRACT

A filtering device having UV light, that includes shell body having a water passage chamber, UV light is disposed in the water passage chamber. A glass tube set is disposed in the water passage chamber around outer periphery of the UV light. The glass tube set includes a first glass tube and a second gas tube both disposed around the outer periphery of the UV light. The lower end of the first glass tube is connected to and acts in cooperation with the lower end of the water passage chamber. The upper end of the first glass tube is separated from the upper end wall of the water passage chamber. The second glass tube is located around the outer periphery of the first glass tube. The lower end of the second glass tube is separated from the lower end wall of the water passage chamber.

### BACKGROUND OF THE INVENTION

### Field of the Invention

The preferred embodiment of the present invention relates to a filtering device, and in particular to a filtering device having an UV light.

### The Prior Arts

Presently on the market, the filtering device having an UV light are already available. The major purpose and function of the UV light is to exterminate various germs in the toilet, and that is realized in the following approaches: 1. utilizing a flow guide block, to disperser the water flow, to disperse the water flow evenly to the metal side wall, so that water flow is turn into vortex; 2. utilizing a water step block to reduce water flow speed, to increase the time the water flow irradiated by the UV light. However, the both approached mentioned above are not capable of exterminate germs in the water, so germs still remain in the toilet.

Therefore, presently, the design and performance of the filtering device having an UV light is not quite satisfactory, and it leaves much room for improvement.

### SUMMARY OF THE INVENTION

In view of the problems and drawbacks of the prior art, the preferred embodiment of the present invention provides a filtering device having UV light, to overcome the shortcomings of the Prior Art.

The embodiment of the present invention provides a filtering device having UV light, that includes shell body having a water passage chamber, UV light is disposed in the water passage chamber. A glass tube set is disposed in the water passage chamber around outer periphery of the UV light.

In an embodiment of the present invention, the glass tube set includes a first glass tube and a second gas tube both disposed around the outer periphery of the UV light. The lower end of the first glass tube is connected to and acts in cooperation with the lower end of the water passage chamber. The upper end of the first glass tube is separated from the upper end wall of the water passage chamber. The second glass tube is located around the outer periphery of the first glass tube. The lower end of the second glass tube is separated from the lower end wall of the water passage chamber. The upper end of the second glass tube is connected to and acts in cooperation with the upper end of the water passage chamber. On the shell body is disposed a water input channel around the outer periphery of the second glass tube

In an embodiment of the present invention, on the shell body is disposed a water output channel around the inner periphery of the first glass tube.

Compared with the Prior, the filtering device having an UV light of the present invention has the following advantages: through using the glass tube set to control the water flow process in the filtering device, so that the water flow can be irradiated by the UV light for longer period of time, to exterminate the germs effectively.

Further scope of the applicability of the present invention and embodiments thereof will become apparent from the detailed descriptions given hereafter. However, it should be understood that the detailed descriptions and specific examples, while indicating preferred embodiments of the present invention, are given by way of illustration only, since various changes and modifications within the spirit and scope of the present invention will become apparent to those skilled in the art from the detailed descriptions.

### BRIEF DESCRIPTION OF THE DRAWINGS

Embodiments of the present invention are described below, by way of example only, with reference to the accompanying drawings in which:

FIG. 1 is a schematic diagram of a filtering device having an UV light according to the present invention.

### DETAILED DESCRIPTION OF THE PREFERRED EMBODIMENT

The purpose, construction, features, functions and advantages of the present invention and its embodiments can be appreciated and understood more thoroughly through the following detailed descriptions with reference to the attached drawings.

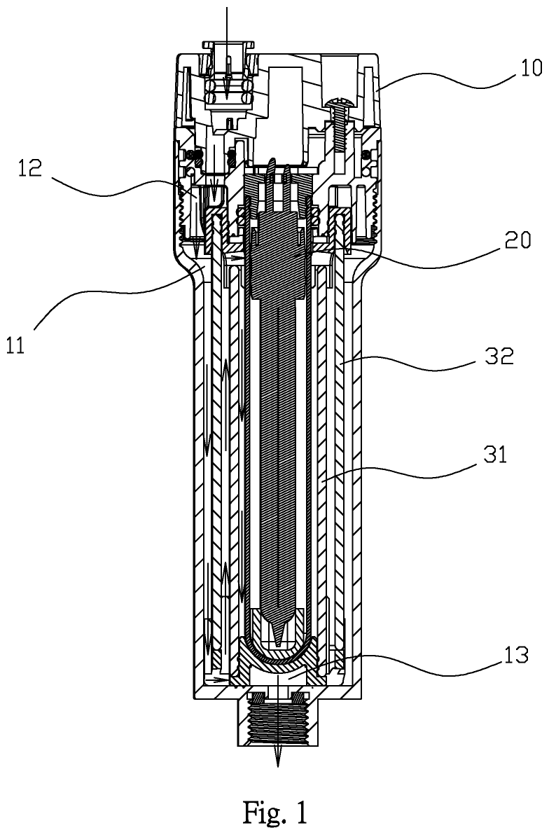

Refer to FIG. 1 for a schematic diagram of a filtering device having an UV light according to the present invention.

As shown in FIG. 1, the embodiment of the present invention provides a filtering device having UV light, that includes shell body 10 having a water passage chamber 11, UV light is disposed in the water passage chamber 11. A glass tube set is disposed in the water passage chamber around outer periphery of the UV light.

In an embodiment of the present invention, the glass tube set includes a first glass tube 31 and a second gas tube 32 both disposed around the outer periphery of the UV light. The lower end of the first glass tube is connected to and acts in cooperation with the lower end of the water passage chamber. The upper end of the first glass tube 31 is separated from the upper end wall of the water passage chamber. The second glass tube is located around the outer periphery of the first glass tube. The lower end of the second glass tube is separated from the lower end wall of the water passage chamber. The upper end of the second glass tube is connected to and acts in cooperation with the upper end of the water passage chamber. On the shell body is disposed a water input channel 12 around the outer periphery of the second glass tube 32.

In an embodiment of the present invention, on the shell body 10 is disposed a water output channel 13 around the inner periphery of the first glass tube 31.

In application, water flows in the shell body, to reach the water passage chamber through the water inlet channel 12. Due to the existence of the glass tube set, water is made to flow from the upper end of the water passage chamber 11, to its lower end. When water reaches the lower end of the water passage chamber 11, the water flows upward between the first glass tube 31 and the second glass tube 32. And then when water reaches the upper end of the water passage chamber 11, and then changes its direction to flow downward. At this time, water flows downward between the first glass tube 31 and the second glass tube 32. Then, water flows out of the shell body 10 through the water output channel 13. The purpose of the present invention is to increase the distance of water flowing through the shell body 10, thus the water is irradiated by the UV light for longer period of time, to exterminate various germs.

The above detailed description of the preferred embodiment is intended to describe more clearly the characteristics and spirit of the present invention. However, the preferred embodiments disclosed above are not intended to be any restrictions to the scope of the present invention. Conversely, its purpose is to include the various changes and equivalent arrangements which are within the scope of the appended claims.

What is claimed is:

1. A filtering device having UV light, that includes shell body having a water passage chamber, the UV light is disposed in the water passage chamber, a glass tube set is disposed in the water passage chamber around an outer periphery of the UV light.

2. The filtering device having UV light as claimed in claim 1, wherein the glass tube set includes a first glass tube and a second gas tube, both disposed around the outer periphery of the UV light, a lower end of the first glass tube is connected to and acts in cooperation with a lower end of the water passage chamber, an upper end of the first glass tube is separated from an upper end wall of the water passage chamber, the second glass tube is located around an outer periphery of the first glass tub, a lower end of the second glass tube is separated from a lower end wall of the water passage chamber, an upper end of the second glass tube is connected to and acts in cooperation with an upper end of the water passage chamber, on the shell body is disposed a water input channel around an outer periphery of the second glass tube, on the shell body is disposed a water output channel around the an inner periphery of the first glass tube.

3. The filtering device having UV light as claimed in claim 2, wherein on the shell body is disposed a water output channel around the inner periphery of the first glass tube
